(19) United States
(12) Patent Application Publication
(10) Pub. No.: US 2016/0015459 A1
(43) Pub. Date: Jan. 21, 2016

(54) Neuro-Surgical Clamp

(71) Applicant: Jeffrey A. Greenberg, Princeton, WV (US)

(72) Inventor: Jeffrey A. Greenberg, Princeton, WV (US)

(21) Appl. No.: 14/698,112

(22) Filed: Apr. 28, 2015

Related U.S. Application Data
(60) Provisional application No. 61/985,183, filed on Apr. 28, 2014.

(51) Int. Cl. A61B 19/00; A61B 17/02

(52) U.S. Cl. CPC A61B 19/203; A61B 19/26; A61B 17/0206; A61B 2019/267; A61B 2017/2808

(57) ABSTRACT

A double clamp whose jaws are mounted on pivots. The clamp finds use in craniotomy procedures where a piece of the skull is removed to expose the brain. The clamp frame of the present invention can be attached to armatures that circle the patient's head. The jaws hold the patient's skull fixed. There are no supporting bars; rather, there is a pair of semi-circular (or other shaped) armatures that partially circle the patient's head and are attached to the frame of the clamp device.

This application is related to and claims priority to U.S. Provisional Patent application No. 61/985,183 filed Apr. 28, 2014. Application 61/985,183 is hereby incorporated by reference in its entirety.

### BACKGROUND

1. Field of the Invention

The present invention relates to clamps used in surgery to secure to the position between a piece of surgical equipment and a patient and more particularly to a neuro-surgical clamp used to secure and steady a retractor during neurosurgery.

2. Description of the Prior Art

Clamps that are used in surgery to hold a piece of surgical equipment in proximity to a patient are known in the art. Position stability is particularly critical in a craniotomy procedure. However, prior art clamps do not adapt to attach to a head clamp or rest to easily support a retractor.

### SUMMARY OF THE INVENTION

The present invention relates to a double clamp whose jaws are mounted on pivots. The clamp finds use in craniotomy procedures where a piece of the skull is removed to expose the brain. The clamp frame of the present invention can be attached to armatures that circle the patient's head. The jaws hold the patient's skull fixed. There are no supporting bars; rather, there is a pair of semi-circular (or other shaped) armatures that partially circle the patient's head and are attached to the frame of the clamp device. These allow the clamping or attachment of retractors and other instruments, all of which are held rigidly steady during the procedure.

### DESCRIPTION OF THE FIGURES

Attention is now directed to several drawings that illustrate features of the present invention:

FIGS. 1A, 1B and 1C show side, top and isometric views of an embodiment of the clamp of the present invention.

FIG. 2 shows details of the use of the clamp of the present invention with armatures.

FIG. 3 shows details of an attachment between the clamp and the armature.

Several drawings and illustrations have been presented to aid in understanding the present invention. The scope of the present invention is not limited to what is shown in the figures.

### DESCRIPTION OF THE PREFERRED EMBODIMENT

The present invention relates to a double clamp whose jaws are mounted on pivots. The clamp finds use in craniotomy procedures where a piece of the skull is removed to expose the brain. The clamp frame of the present invention can be attached to armatures that circle the patient's head. The jaws hold the patient's skull fixed. There are no supporting bars; rather, there is a pair of semi-circular (or other shaped) armatures that partially circle the patient's head and are attached to the frame of the clamp device.

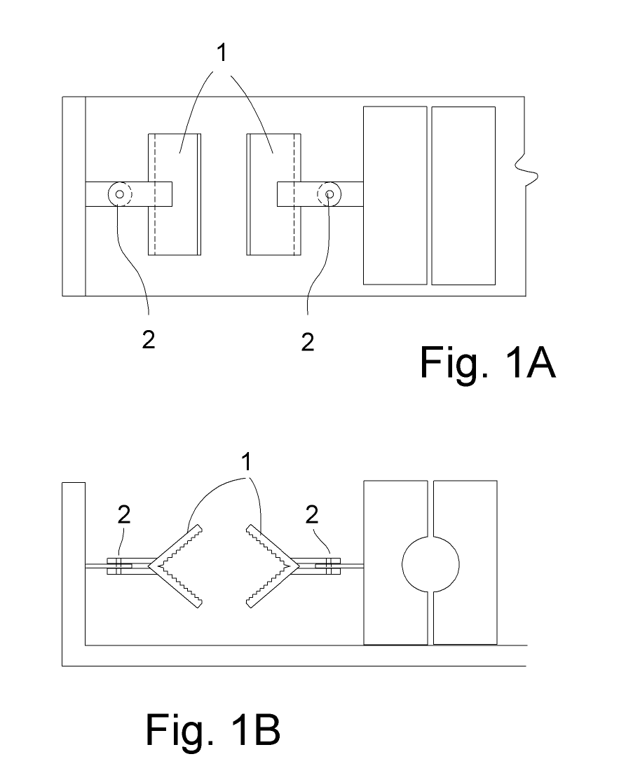

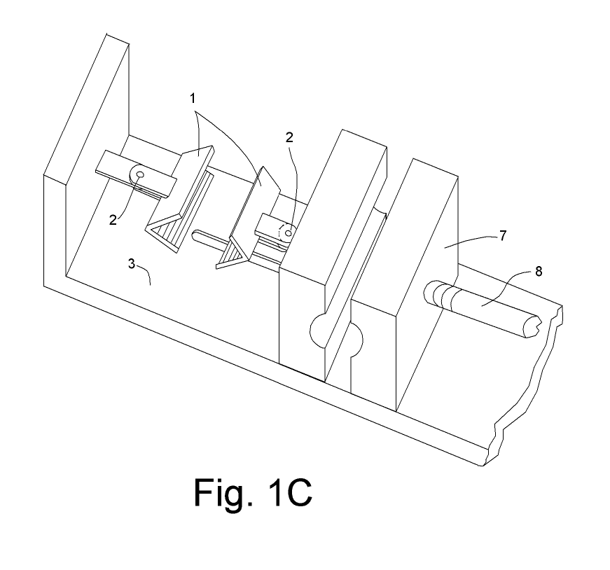

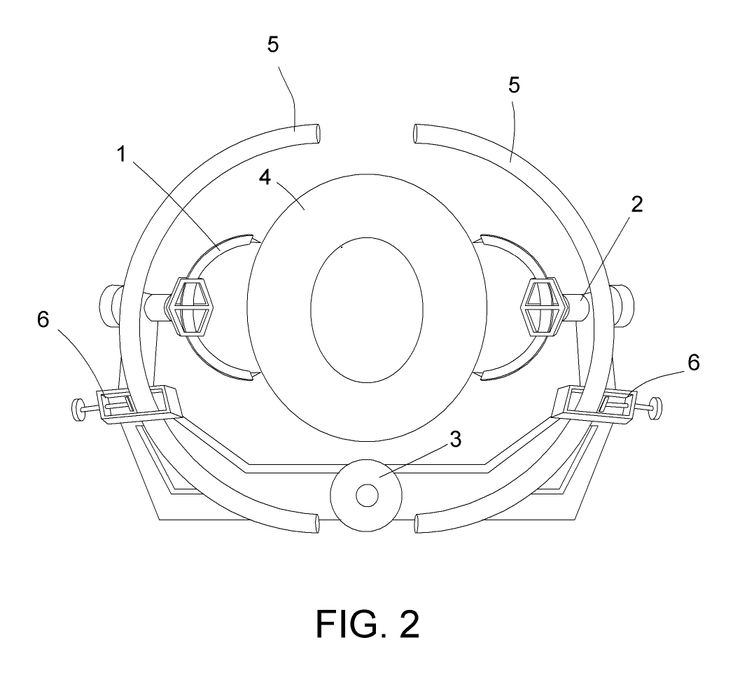

FIGS. 1A-1C show an embodiment of the clamp device. Jaws 1 are attached to a frame 3 by pivot joints 2. This allows the jaws 1 to freely pivot to attain the optimum position with respect to the patient's head 4 (FIG. 2). The pivot joints 2 are further attached to a frame 3 which can be wood, metal or any other rigid material. A movable block 7 can be screwed by a threaded rod 8 to tighten the jaws as shown in FIG. 1C. The jaws are adapted to contact the patient's head during surgery.

FIG. 2 shows an entire assembly schematically. The clamp frame 3 is attached to pair of semi-circular armatures 5 which partially encircle the patient's head 4. The armatures 5 can be metal or other rigid material. The attachment can be made through adjustable smaller clamps 6 or by any other attachment method that will allow secure attachment of that armatures 5 to the clamp frame 3. The armatures are used to hold surgical instruments. The armatures can be flat or other shape. The preferred shape is flat with a thickness smaller than their width. These typically take the shape of part of a semi-circle that is about twice the diameter of the patient's head. The armatures circle the head and provide a rigid support for the clamped or otherwise attached medical instruments. In an alternate embodiment, a single armature that is almost a full circle may be used.

The preferred material for the armatures is stainless steel; however, any rigid material may be used including other metals and hard plastics. The armatures must hold clamped medical instruments in place during a procedure without flexing or otherwise distorting or moving.

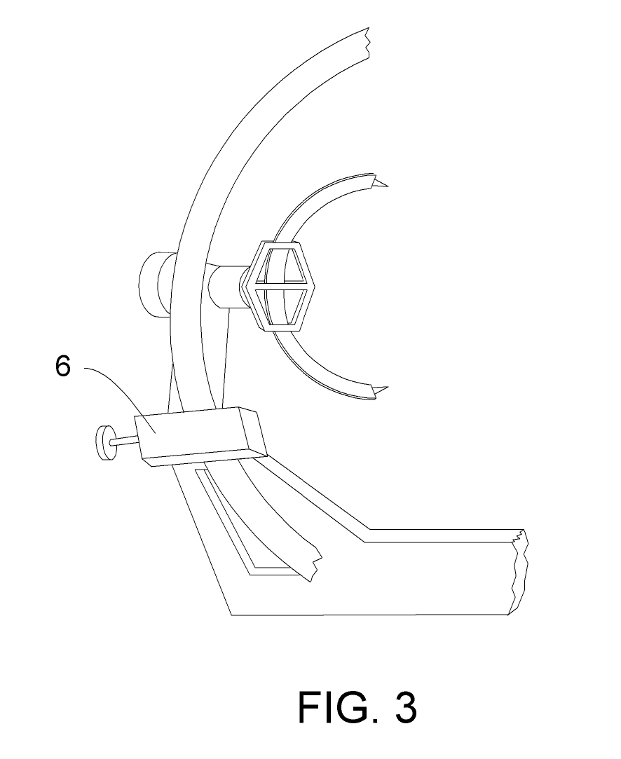

FIG. 3 shows a detail of an embodiment of a smaller removable clamp 6 used to attach the clamp unit of the invention to the armatures.

The present invention allows a retractor or other surgical instrument to be held in proximity to the patient's head in a totally fixed position typically during a craniotomy procedure. The instrument is held continuously steady during surgery. The retractor is typically clamped or otherwise attached to the armature during the procedure. The arrangement is simple enough that it is easy to set up and easy to rearrange.

Several drawings and illustrations have been presented to aid in understanding the present invention. One with skill in the art will realize that numerous changes and variations may be made without departing from the spirit of the invention. Each of these changes and variations is within the scope of the present invention.

I claim:

1. A neuro-surgical clamp device comprising:
a clamp frame;
a pair of opposing adjustable jaws pivotably mounted to the clamp frame; the jaws adapted to clamp a patient's head during neurosurgery;
a pair of substantially semi-circular armatures with diameters greater than a patient's head clampable to the jaws or clamp frame;
whereby, surgical instruments can be attached to the armatures during a surgical procedure.

2. The neuro-surgical clamp device of claim 1 wherein the armatures are clamped to the jaws using removable clamps.

3. The neuro-surgical clamp device of claim 1 wherein the armatures have a flat surface of smaller dimension than their width.

4. The neuro-surgical clamp device of claim 1 wherein the armatures are stainless steel.

5. A neuro-surgical clamp device that includes a pair of vise jaws adapted to be tightened on a patient's head during neuro-surgery, and that includes a pair of flat, semi-circular members attachable to the jaws that circle a region around where a patient's head would be during surgery, the semi-circular members configured to hold clamped medical instruments.

6. The neuro-surgical clamp device of claim 5 wherein the semi-circular members attached to the jaws with removable clamps.
